(19) United States
(12) Patent Application Publication
(10) Pub. No.: US 2004/0214833 A1
(43) Pub. Date: Oct. 28, 2004

(54) Tricyclic heteroaromatic compounds

(75) Inventors: Norbert Hauel, Schemmerhofen (DE); Henning Draheim, Warthausen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/805,860

(22) Filed: Mar. 22, 2004

Related U.S. Application Data
(60) Provisional application No. 60/465,161, filed on Apr. 24, 2003.

(30) Foreign Application Priority Data
Apr. 11, 2003 (DE) 10316659

(51) Int. Cl. C07D 498/02; C07D 491/02

(52) U.S. Cl. 514/255.05; 514/291; 514/292; 546/82; 514/256; 544/333; 544/405; 514/227.8; 514/234.2

(57) ABSTRACT

The invention relates to tricyclic heteroaromatic compounds of general formula (I) and the salts thereof

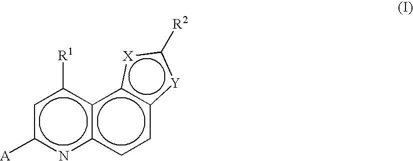

wherein the structural elements X, Y, A, R¹ and R² are defined in the specification. The invention also relates to the preparation of these compounds and their use as pharmaceutical compositions, particularly as analgesics.

The invention relates to tricyclic heteroaromatic compounds and the salts thereof, their preparation and their use as pharmaceutical compositions, particularly as analgesics.

Acute pain, i.e. brief transient pain, usually dies away rapidly once the cause has been eliminated and gives rise to generally negligible damage to the tissues. However, pain may also last for a longer period. This is then known as chronic pain which is generally associated with tissue damage, inflammation or other problems. Complaints accompanied by chronic or chronically recurring pain include, inter alia, migraine, neuralgia, muscle pain and inflammatory pain. The chronic neuronal pains include inter alia post-operative pain, shingles, phantom pain, diabetic neuropathy, pain after chronic nerve compression as well as end-stage Aids and cancer.

A distinction is made between primary pain, also known as sharp pain, and secondary pain, so-called dull pain. Primary pain is experienced as immediate pain on injury. It is transmitted to the brain at high speed (about 20 metres per second). If injuries do not cause primary pain, secondary pain is felt. This reaches the brain much more slowly (about two metres per second) but is more persistent and remains as a dull pain for a longer period.

To treat mild pain or headaches there are active substances available such as acetylsalicylic acid, paracetamol or ibuprofen. Particularly severe pain is treated with opium-related agents such as codeine, morphine or similar substances. Their task is primarily to improve the patient's quality of life by suppressing the pain.

Opiates and opioids act predominantly on the central nervous system. In addition to their pain-inhibiting activity they may also have a sedative (calming) effect or give a feeling of euphoria, inhibit the respiratory centre and suppress coughing. They include substances such as codeine, morphine, tilidine and tramadol.

Non-opioid analgesics generally act on the peripheral nervous system and also have an antipyretic and anti-inflammatory effect. Often an additional stimulant such as caffeine is added to the active substances. Examples of such analgesics are Doppel-Spalt, Eudorlin, Migränin, Neuralgin, Thomapyrin, Titralgan and Vivimed.

N-type calcium channel antagonists for the treatment and prevention of pain are described in International Applications WO 02/36567, WO 02/36568 and WO 02/36569.

The present invention provides new compounds and the salts thereof which are suitable for relieving pain, particularly chronic pain. These compounds are described by general formula (I):

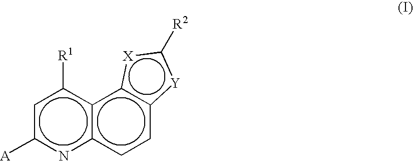

In this formula (I)

X denotes a nitrogen atom (N), oxygen atom (O) or sulphur atom (S);

Y denotes a nitrogen atom, if X denotes an oxygen atom or sulphur atom;

Y denotes a nitrogen atom with a bound group R³ or a sulphur atom or an oxygen atom, if X denotes a nitrogen atom;

A denotes an unsubstituted or substituted mono-, di- or tricyclic aromatic group, which contains either no or 1-3 heteroatoms selected from nitrogen, oxygen and sulphur, at least one of the heteroatoms being a nitrogen atom;

R¹ denotes hydroxy, fluorine, chlorine or bromine, amino, (C₁₋₆)alkylamino, di(C₁₋₆)alkylamino, (C₃₋₇)cycloalkylamino, di(C₃₋₇)cycloalkylamino, (C₁₋₆)alkyl-(C₃₋₇)cycloalkylamino, as well as the heterocycloalkyl groups acetidin-1-yl, pyrrolidin-1-yl, piperidin-1-yl, morpholin-4-yl, thiomorpholin-4-yl, thiomorpholin-S-oxid-4-yl, thiomorpholin-S-dioxid-4-yl, or hexamethyleneimino;

R² and R³ independently of one another denote hydrogen (H), (C₁₋₈)alkyl or (C₃₋₇)cycloalkyl; and

the term “alkyl” describes both saturated and also mono- or polyunsaturated aliphatic hydrocarbon radicals. By (Cₙ₋ₘ)alkyl groups are meant those which contain n to m carbon atoms, where n and m denote whole numbers. Unless otherwise defined saturated radicals are preferably (C₁₋₁₀)alkyl groups, while unsaturated radicals are preferably (C₂₋₁₂)alkyl groups.

At the same time the term “alkyl” includes both straight-chain and branched hydrocarbon radicals. Unless otherwise defined straight-chain radicals are preferably (C₁₋₈)alkyl groups, branched radicals are preferably (C₃₋₁₀)alkyl groups.

Hydrogen atoms of alkyl radicals may be partly or totally replaced by halogen atoms. Examples of such halogen atoms are the fluorine, chlorine, bromine and iodine atoms. If all the hydrogen atoms are replaced by halogens, this is referred to as a “perhaloalkyl” radical.

The term “cycloalkyl” covers saturated and mono- or polyunsaturated aliphatic hydrocarbon radicals which form cyclic carbon chains, i.e. carbon chains closed in a ring, which do not constitute an aromatic ring system. The term (Cₙ₋ₘ)cycloalkyl groups denotes those wherein the ring structure is formed by n to m carbon atoms, where n and m represent whole numbers greater than 2. Unless otherwise defined they are preferably monocyclic (C₃₋₈)cycloalkyl groups.

The term “heterocycloalkyl” describes cycloalkyl radicals whose closed chain contains one or more heteroatoms in addition to carbon atoms. These heteroatoms may be nitrogen, oxygen or sulphur atoms. The term (Cₙ₋ₘ)heterocycloalkyl groups denotes those whose ring structure is formed by n to m atoms, where n and m represent whole numbers which are greater than 3. Unless otherwise stated, monocyclic (C₄₋₈) and bicyclic (C₈₋₁₁)heterocycloalkyl groups are preferred. Each ring may usually contain 1 to 4 heteroatoms. The attachment of the group to the compound according to the invention may take place via each carbon or heteroatom of the ring system, which allows the formation of a stable bond. Examples of “heterocycloalkyl” radicals are pyrrolinyl, pyrrolidinyl, pyrazolinyl, pyrazolidinyl, piperidinyl, morpholinyl, thiomorpholinyl, piperazinyl, indolinyl, azetidinyl, tetrahydropyranyl, tetrahydrothiopyranyl, tetrahydrofuranyl, hexahydropyrimidinyl, hexahydropyridazinyl, dihydrooxazolyl, 1,2-thiazinanyl-1,1-dioxide, 1,2,6-thiadiazinanyl-1,1-dioxide, isothiazolidinyl-1,1-dioxide and imidazolidinyl-2,4-dione.

The term “acyl” describes both saturated and mono- or polyunsaturated radicals of aliphatic carboxylic acids, which are formed by the elimination of the OH group from the carboxy group. By (Cₙ₋ₘ)acyl groups are meant those which contain n to m carbon atoms, where n and m denote whole numbers. Unless otherwise stated the saturated radicals are preferably (C₁₋₁₀)acyl groups, while the unsaturated radicals are preferably (C₂₋₁₂)acyl groups. At the same time the term “acyl” denotes both straight-chain and branched radicals. Straight-chain radicals are preferably (C₁₋₈)acyl groups, branched radicals are preferably (C₃₋₁₀)acyl groups. Hydrogen atoms of these radicals may be partly or totally replaced by halogen atoms. Examples of this are the fluorine, chlorine, bromine and iodine atoms. If all the hydrogen atoms are replaced by halogen atoms, the radical is referred to as a “perhaloacyl” radical.

Terms made up of syllables or functional groups whose meaning is well known from the specialist literature, and one or more of the syllables defined above, refer to radicals composed of the corresponding structural elements. Thus, the terms “alkyloxy” and “alkylthio” denote alkyl groups which are bound via an oxygen or sulphur atom to another structural element. An “alkylcarbonyl” radical represents an alkyl group which is bound via a carbonyl group (C═O) to another structural element. In an “acylamino” group one of the hydrogen atoms of an amino group is replaced by an acyl group.

In selected embodiments of the compound according to the invention both X and Y denote a nitrogen atom.

In other embodiments the group A denotes phenyl, pyridyl, pyrimidyl, pyridazinyl, pyrazinyl, imidazolyl, pyrazolyl, oxazolyl, isoxazolyl, furazanyl, thiazolyl, isothiazolyl, or pyrrolyl, which may be unsubstituted or substituted by the groups R⁴, R⁵ and R⁶ wherein R⁴, R⁵ and R⁶ independently of one another denote hydrogen (H), (C₁₋₈)alkyl, monofluoro(C₁₋₅)alkyl, difluoro(C₁₋₅)alkyl, trifluoro(C₁₋₅)alkyl, (C₃₋₇)cycloalkyl, hydroxy, (C₁₋₆)alkoxy, fluoromethyloxy, difluoromethyloxy, trifluoromethyloxy, (C₃₋₆)cycloalkyloxy, fluorine, chlorine, bromine, carboxy, (C₁₋₆)alkoxycarbonyl, amino, (C₁₋₆)alkylamino, di(C₁₋₆)alkylamino, acetidin-1-yl, pyrrolidin-1-yl, piperidin-1-yl, (C₁₋₄)acylamino, (C₁₋₆)alkyl-(C₁₋₄)acylamino, aminocarbonyl, (C₁₋₆)alkylaminocarbonyl, di(C₁₋₆)alkylaminocarbonyl, acetidin-1-yl-carbonyl, pyrrolidin-1-yl-carbonyl or piperidin-1-yl-carbonyl. The group A is preferably a phenyl or pyridyl group substituted by 1 to 3 substituents.

In yet other embodiments the group R¹ denotes amino, (C₁₋₆)alkylamino, di(C₁₋₆)alkylamino, (C₃₋₇)cycloalkylamino, di(C₃₋₇)cycloalkylamino or (C₁₋₆)alkyl-(C₃₋₇)cycloalkylamino. Preferred groups of R¹ are in particular those which have the properties of an electron donor.

Preferred groups R² and R³ are hydrogen, (C₁₋₆)alkyl or (C₃₋₆)cycloalkyl. Preferred groups R⁴, R⁵ and R⁶ are hydrogen, fluorine, chlorine, bromine, (C₁₋₃)alkyl, fluoromethyl, difluoromethyl, trifluoromethyl, fluoromethyloxy, difluoromethyloxy, trifluoromethyloxy and di(C₁₋₃)alkylamino.

The invention thus also includes pharmaceutically suitable derivatives of the compounds of formula (I). By “pharmaceutically suitable derivatives” are meant salts and precursors of the compounds of formula (I), which after administration to a patient are converted directly or indirectly into one of the compounds according to the invention or one of the pharmacologically active metabolites thereof. These are above all salts, acids and esters of the compounds according to the invention. Of particular importance are salts which are derived from pharmaceutically suitable inorganic or organic acids or bases. Examples include the salts with acids such as hydrochloric acid, hydrobromic acid, sulphuric acid, phosphoric acid, nitric acid, oxalic acid, malonic acid, fumaric acid, maleic acid, tartaric acid, citric acid, ascorbic acid and methanesulphonic acid.

Precursors are compounds which, after a simple chemical conversion, yield compounds of formula (I) or one of the pharmacologically active metabolites thereof. Simple chemical conversions include hydrolysis, oxidation and reduction which may occur e.g. enzymatically or metabolically. For the present invention this means that administering a precursor of the compounds according to the invention to a patient leads to the conversion of this precursor into a compound of formula (I), which then produces the desired pharmacological effect.

Compounds according to this invention which have one or more asymmetric carbon atoms may occur as racemates or racemic mixtures, as isolated enantiomers, as diastereomeric mixtures or as individual diastereomers. Each stereogenic carbon atom may be present in the R or S configuration or in a combination of the two configurations. Some of the compounds may also be present in tautomeric forms.

The compounds according to the invention may for example be prepared according to the following reaction plan:

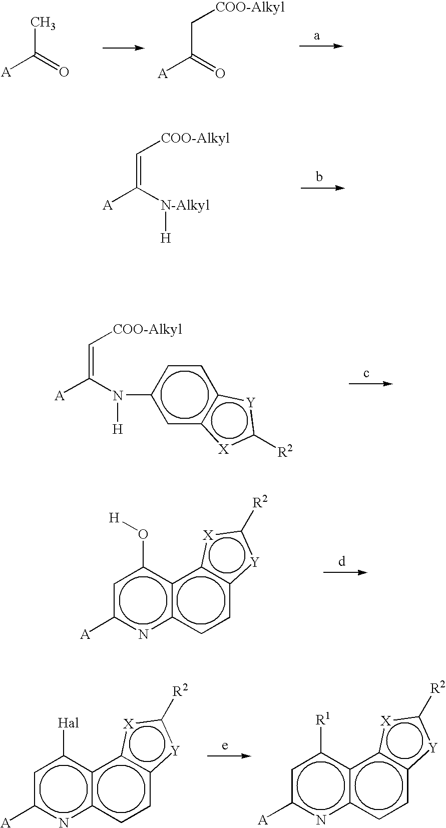

A 3-oxo-propionic acid ester which may be prepared from the corresponding acetyl derivative, the carbonyl group of which is bound to the desired group A, is reacted, for example, with the salt of a primary amine such as N-methylammonium acetate to form the corresponding acrylic acid ester derivative. The latter is then reacted with the desired amino derivative of benzimidazole, benzoxazole or benzthiazole. The group introduced by the primary amine is replaced by the corresponding radical of the benzimidazole, benzoxazole or benzthiazole derivative. Subsequently, cyclisation is carried out by heating in a suitable solvent to obtain the corresponding derivative of 3H-imidazo[4,5-f]quinolines, 3H-oxazo[4,5-f]quinolines or 3H-thiazo[4,5-f]quinolines. The compound obtained is hydroxylated at position 9 and may be halogenated at this position using compounds such as phosphorus oxychloride before being reacted with the desired amine in a last step to form a compound according to the invention. The intermediate products obtained according to the individual process steps are purified if necessary.

The compounds thus prepared are valuable as active substances for pharmaceutical compositions, particularly for preparing an analgesic for alleviating or treating pain. This activity can be determined using a simple test procedure in which the pain reactions of animals are observed and quantitatively evaluated. For this, the following procedure is carried out with the compounds according to the invention:

Male rats (strain: Chbb-THOM; weight: 200 to 300 g) are injected with 20 μL of a 2% formaldehyde solution into the plantar region of the right hind paw. Immediately afterwards the number of flinches (spasms of the affected hind paw) and the time spent licking the affected paw are recorded over a period of one hour. After five minutes in each case the values are collected into epochs and from these values time/activity curves are plotted for the flinches and licking. Typically two phases of the formalin activity (flinches, licking) are observed: A first phase from 0 to 10 minutes and a second phase from 10 to 60 minutes. After the first phase the number of flinches and the time spent licking fall towards zero (intermediate phase). From the time/activity curves the areas under the curves for the first and second phase are determined. As a rule, five animals each are used as control, for the administration of placebo and for receiving the dose of substance. The results of giving the doses of substance are compared with those of the control and ED₅₀ values are thus determined. The ED₅₀ is the dose at which the control values are inhibited by 50%.

The antinociceptive activity of the compounds of this invention is based on a blockade of the voltage-dependent N-type calcium channels. This inhibiting activity is detected electrophysiologically by the Patch-Clamp technique (cf: Improved patch-clamp techniques for high resolution current recording from cells and cell-free membrane patches; Hamill et al.; Pflügers Archiv, 391, 1981, 85-100) on recombinant HEK 293 cells which express the N-type calcium channel. Thus, in these investigations, for example, the compounds of Examples 4 and 6 exhibited IC₅₀ values of 3.6 and 2.0 μmol/L, respectively.

Thus the compounds according to the invention may be used in procedures intended to alleviate or treat pain in which a patient is given a therapeutically effective amount of the compound according to the invention. The pain treated may be acute pain, chronic pain, neuropathic pain or post-operative pain, as well as pain associated with migraine, arthralgia, neuropathies, nerve damage, diabetic neuropathy, neurodegeneration, neurotic skin diseases, stroke, hypersensitive bladder, irritable bowel, respiratory complaints such as asthma or chronic obstructive pulmonary disease, irritations of the skin, eyes or mucous membranes, duodenal and gastric ulcers, gastric inflammation or other inflammatory diseases.

For treating pain it may be advantageous to combine the compounds according to the invention with stimulants such as caffeine or other pain-relieving active substances. If active substances are available for treating the cause of the pain, these may be combined with the compounds according to the invention. If further medical treatment is indicated, quite apart from the pain relief, e.g. to treat high blood pressure or diabetes, the active substances needed for such treatment may also be combined with the compounds according to the invention.

The dosage required to achieve a pain-relieving activity is conveniently 0.01 to 3 mg/kg body weight, preferably 0.1 to 1 mg/kg body weight when administered intravenously, and 0.1 to 8 mg/kg body weight, preferably 0.5 to 3 mg/kg body weight when administered orally, in each case 1 to 3 times a day. For this the compounds of formula (I) prepared according to the invention, optionally in combination with other active substances, may be incorporated together with one or more inert conventional carriers and/or diluents, e.g. with maize starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetylstearylalcohol, carboxymethylcellulose or fatty substances such as hard fat or suitable mixtures thereof, in conventional galenic preparations such as tablets, coated tablets, capsules, powders, suspensions or suppositories.

Compounds according to the invention are described in the examples that follow. The skilled man will be aware that these Examples serve to illustrate the subject matter of the invention and are not intended to restrict the general technical teaching of the invention provided.

### EXAMPLES

### Example 1

### 3-methyl-9-methylamino-7-(pyridin-4-yl)-3H-imidazo[4,5-f]quinoline

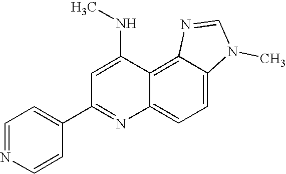

### 1a. ethyl 3-methylamino-3-(pyridin-4-yl)-acrylate

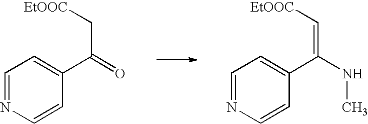

A solution of 17.2 g (89 mmol) of ethyl 3-oxo-3-(pyridin-4-yl)-propionate and 41.0 g (450 mmol) of N-methylammonium-acetate in 120 ml of ethanol is refluxed for one hour, then the solvent is evaporated off. The residue is dissolved in approx. 300 ml dichloromethane, this solution is washed twice with approx. 100 ml of water, dried over sodium sulphate and then concentrated by evaporation. The product thus obtained is further processed without any further purification.

Yield: 98% of theory. C₁₁H₁₄N₂O₂ (206.25) R_{f} value: 0.40 (silica gel, petroleum ether/ethyl acetate 1:1)

Mass spectrum:

(M+H)⁺=207

(M−H)⁻=205

### 1b. ethyl 3-(1-methyl-1H-benzimidazol-5-yl-amino)-3-(pyridin-4-yl)-acrylate

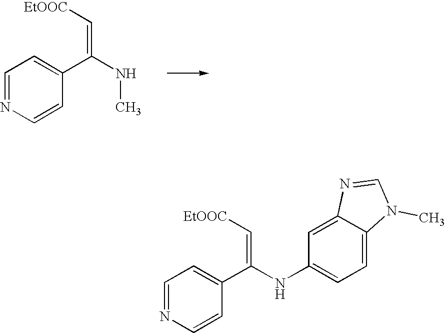

A solution of 619 mg (3.0 mmol) of ethyl 3-methylamino-3-(pyridin-4-yl)-acrylate and 442 mg (3.0 mmol) of 5-amino-1-methyl-benzimidazole in a mixture of 36 ml dichloromethane and 4 ml of ethanol is refluxed for approx. 20 hours. Then it is evaporated to dryness and the crude product thus obtained is purified by column chromatography (silica gel; eluant: dichloromethane with 2-5% ethanol).

Yield: 26% of theory. C₁₈H₁₈N₄O₂ (322.37) R_{f} value: 0.22 (silica gel, dichloromethane/ethanol 19:1)

### 1c. 9-hydroxy-3-methyl-7-(pyridin-4-yl)-3H-imidazo[4,5-f]quinoline

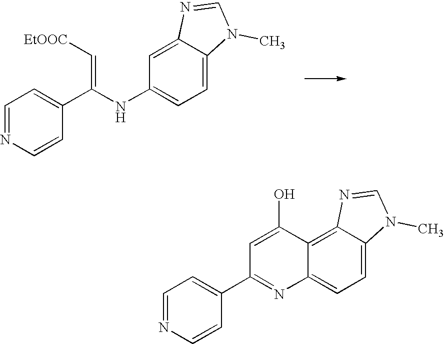

1.7 g (5.27 mmol) of ethyl 3-(1-methyl-1H-benzimidazol-5-yl-amino)-3-(pyridin-4-yl)-acrylate are added batchwise to 20 ml Dowtherm (Sigma-Aldrich Chemie GmbH, D-82024 Taufkirchen, Germany), which has been heated to 250° C. with stirring, and the mixture is stirred for a further hour at 250° C. The mixture is then cooled to ambient temperature, diluted with approx. 30 ml petroleum ether, the precipitated product is filtered off, washed again with approx. 30 ml petroleum ether and dried.

Yield: 82% of theory. C₁₆H₁₂N₄O (276.30) R_{f} value: 0.13 (silica gel; dichloromethane/ethanol 9:1)

Mass spectrum:

(M+H)⁺=277

(M−H)⁻=275

¹H-NMR spectrum (d₆-DMSO): δ=3.95 (s, 3H); 6.42 (s, 1H); 7.90 (d, 2H); 8.03 (s, 1H); 8.30 (s, 1H); 8.47 (s, 1H); 8.81 (d, 2H); 11.77 (s, 1H) ppm.

### 1d. 9-chloro-3-methyl-7-(pyridin-4-yl)-3H-imidazo[4,5-f]quinoline

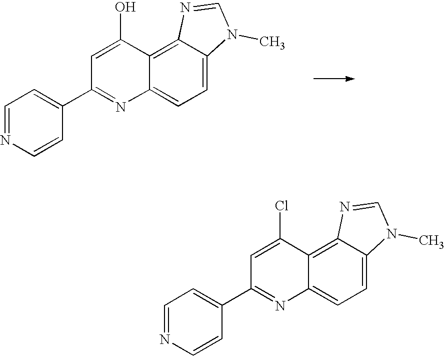

1.2 g (276 mmol) of 9-hydroxy-3-methyl-7-(pyridin-4-yl)-3H-imidazo[4,5-f ]quinoline are stirred in 15 ml phosphorus oxychloride for one hour at 50° C. Then the phosphorus oxychloride is distilled off in vacuo and the residue is neutralised with saturated sodium hydrogen carbonate solution. The precipitated solid is suction filtered, dissolved in a mixture of dichloromethane and ethanol (9:1), the solution is filtered and evaporated down again. The product is thus obtained as a crystalline solid.

Yield: 23% of theory. C₁₆H₁₁ClN₄ (294.75) R_{f} value: 0.59 (silica gel; dichloromethane/ethanol 9:1) ¹H-NMR spectrum (d₆-DMSO): δ=4.04 (s, 3H); 8.31 (d, 2H); 8.36 (s, 1H); 8.48 (s, 1H); 8.50 (s, 1H); 8.16 (s, 1H); 8.80 (d, 2H) ppm.

### 1e. 3-methyl-9-methylamino-7-(pyridin-4-yl)-3H-imidazo[4,5-f]quinoline

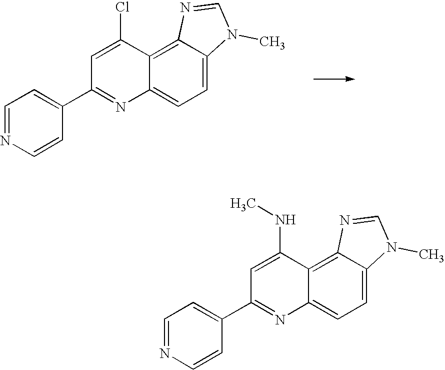

A 33% solution of methylamine in ethanol (3 ml) is diluted with 15 ml of ethanol, then 270 mg (0.92 mmol) of 9-chloro-3-methyl-7-(pyridin-4-yl)-3H-imidazo [4,5-f]quinoline are added and this mixture is heated to 120° C. for 6 hours in a Roth bomb. It is then evaporated to dryness and the crude product thus obtained is purified by column chromatography (silica gel; eluant: dichloromethane with 2-7% ethanol).

Yield: 17% of theory. C₁₇H₁₅N₅ (289.34) R_{f} value: 0.51 (silica gel; dichloromethane/ethanol 9:1) ¹H-NMR spectrum (d₆-DMSO): δ=3.20 (d, 3H); 4.02 (s, 3H); 7.20 (s, 1H); 7.82 (d, 1H); 7.99 (d, 1H); 8.20 (d, 2H); 8.46 (s, 1H); 8.72 (d, 2H); 8.92 (s, 1H) ppm. IC₅₀ value: 9.9 μM

### Example 2

### 7-(3-fluorophenyl)-3-methyl-9-methylamino-3H-imidazo [4,5-f]quinoline

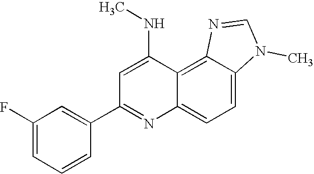

This is prepared analogously to Example 1, with 9-chloro-7-(3-fluorophenyl)-3-methyl-3H-imidazo[4,5-f]quinoline being reacted in ethanolic methylamine solution in the last process step 1e.

Yield: 41% of theory. C₁₈H₁₅FN₄ (306.35) Mass spectrum: (M+H)⁺=307 ¹H-NMR spectrum (d₆-DMSO): δ=3.20 (d, 3H); 4.01 (s, 3H); 7.12 (s, 1H); 7.28 (dt, 1H); 7.55 (q, 1H); 7.80 (d, 1H); 7.92 (d, 1H); 8.08 (dt, 1H); 8.12 (d, 1H), 8.41 (s, 1H); 8.73 (q, 1H) ppm. IC₅₀ value: 15.2 μM

### Example 3

### 9-dimethylamino-7-(3-fluorophenyl)-3-methyl-3H-imidazo [4,5-f]quinoline

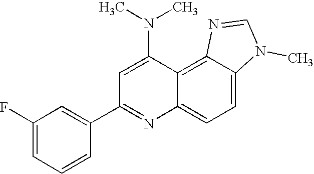

This is prepared analogously to Example 1, with 9-chloro-7-(3-fluorophenyl)-3-methyl-3H-imidazo[4,5-f]quinoline being reacted in ethanolic dimethylamine solution in process step 1e.

Yield: 25% of theory. C₁₉H₁₇FN₄ (320.37) Mass spectrum: (M+H)⁺=321 ¹H-NMR spectrum (d₆-DMSO): δ=3.08 (s, 6H); 3.99 (s, 3H); 7.30 (dt, 1H); 7.50 (s, 1H); 7.58 (q, 1H); 7.90 (d, 1H); 7.99 (d, 1H); 8.05-8.12 (m, 2H); 8.33 (s, 1H) ppm. IC₅₀ value: biphasic

### Example 4

### 9-dimethylamino-7-(3-fluorophenyl)-2-methyl-thiazolo[4,5-f]quinoline

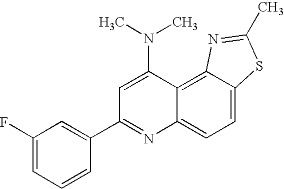

This is prepared analogously to Example 1, with 9-chloro-7-(3-fluorophenyl)-2-methyl-thiazolo[4,5-f]quinoline being reacted in ethanolic dimethylamine solution in process step 1e.

Yield: 36% of theory. C₁₉H₁₆FN₃S (337.42)

Mass spectrum: (M+H)⁺⁼338 ¹H-NMR spectrum (d₆-DMSO): δ=2.92 (s, 3H); 3.05 (s, 6H); 7.32(dt, 1H); 7.51(s, 1H); 7.59(q, 1H); 7.95(d, 1H); 8.05-8.15 (m, 2H); 8.30 (d, 1H) ppm. IC₅₀ value:3.6 μM

### Example 5

### 9-dimethylamino-7-(3-fluorophenyl)-thiazolo[5,4-f]quinoline

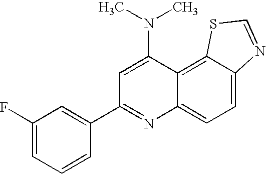

This is prepared analogously to Example 1, with 9-chloro-7-(3-fluorophenyl)-thiazolo [5,4-f]quinoline being reacted in ethanolic dimethylamine solution in process step 1e.

Yield: 52% of theory. C₁₈H₁₄FN₃S (323.39) Mass spectrum: (M+H)⁺=324 ¹H-NMR spectrum (d₆-DMSO): δ=2.91 (s, 6H); 7.36 (dt, 1H); 7.61 (q, 1H); 8.02 (s, 1H); 8.13-8.22 (m, 3H); 8.40 (d, 1H); 9.52 (s, 1H) ppm. IC₅₀ value: 14.3 μM

### Example 6

### 7-(3-fluorophenyl)-2-methyl-9-methylamino thiazolo[4,5-f]quinoline

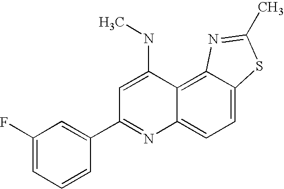

This is prepared analogously to Example 1, with 9-chloro-7-(3-fluorophenyl)-2-methyl-thiazolo[4,5-f]quinoline being reacted in ethanolic methylamine solution in process step 1e.

Yield: 56% of theory. C₁₈H₁₄FN₃S (323.39) R_{f} value:0.48 (silica gel; dichloromethane/methanol 9:1) Mass spectrum: (M+H)⁺=324 ¹H-NMR spectrum (d₆-DMSO): δ=2.97 (s, 3H); 3.18 (d, 3H), 7.14 (s, 1H); 7.30 (dt, 1H); 7.57 (q, 1H); 7.88 (d, 1H); 8.05-8.15 (m, 2H); 8.23 (d, 1H); 9.13 (q, 1H) ppm. IC₅₀ value: 2.0 μM

### Example 7

### 9-dimethylamino-3-methyl-7-(pyridin-3-yl)-3H-imidazo[4,5-f]quinoline

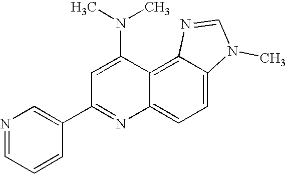

This is prepared analogously to Example 1, with 9-chloro-3-methyl-3H-7-(pyridine3-yl)-imidazo[4,5-f]quinoline being reacted in ethanolic dimethylamine solution in process step 1e.

Yield: 7.6% of theory. C₁₈H₁₇N₅ (303.37)

Mass spectrum:

(M⁺=303

(M+H)⁺=304

### Example 8

### 3-methyl-9-methylamino-7-(pyridin-3-yl)-3H-imidazo[4,5-f]quinoline

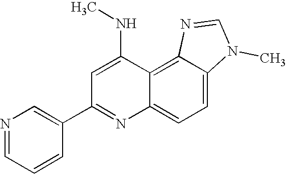

This is prepared analogously to Example 1, with 9-chloro-3-methyl-7-(pyridin-3-yl)-3H-imidazo[4,5-f]quinoline being reacted in ethanolic methylamine solution in process step 1e.

Yield: 15% of theory. C₁₇H₁₅N₅ (289.34) Mass spectrum: (M+H)⁺=290 ¹H-NMR spectrum (d₆-DMSO): δ=3.20 (d, 3H); 4.01 (s, 3H); 7.15 (s, 1H);

7.53 (m, 1H); 7.81 (d, 1H); 7.94 (d, 1H); 8.41 (s, 1H); 8.58 (dt, 1H); (d, 1H); 8.80 (m, 1H); 9.41 (s, 1H) ppm.

### Example 9

### 2-methyl-9-methylamino-7-(pyridin-3-yl)-thiazolo[4,5-f]quinoline

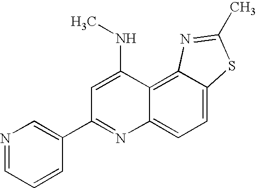

This is prepared analogously to Example 1, with 9-chloro-2-methyl-7-(pyridin-3-yl)-thiazolo[4,5-f]quinoline being reacted in ethanolic methylamine solution in process step 1e.

Yield: 19% of theory. C₁₇H₁₄N₄S (306.37) Mass spectrum: (M+H)⁺=307 ¹H-NMR spectrum (d₆-DMSO): δ=2.99 (s, 3H); 3.20 (d, 3H); 7.20 (s, 1H); 7.54 (m, 1H); 7.90 (d, ₁H); 8.27 (d, 1H); 8.60 (dt, 1H); 8.65 (d, 1H); 9.20 (m, 1H); 9.42 (s, 1H) ppm.

### Example 10

### 9-dimethylamino-2-methyl-7-(pyridin-3-yl)-thiazolo[4,5-f]quinoline

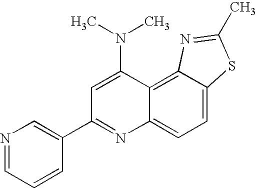

This is prepared analogously to Example 1, with 9-chloro-2-methyl-7-(pyridin-3-yl)-thiazolo[4,5-f]quinoline being reacted in ethanolic dimethylamine solution in process step 1e.

Yield: 14% of theory. C₁₈H₁₆N₄S (320.42) Mass spectrum: (M+H)⁺=321 ¹H-NMR spectrum (d₆-DMSO): δ=2.92 (s, 3H); 3.05 (s, 6H); 7.05 (s, 1H); 7.08 (m, 1H); 7.96 (d, 1H); 8.31 (d, 1H); 8.61 (dt, 1H); 8.68 (m, 1H); 9.43 (s, 1H) ppm.

What is claimed is:

1. A compound of formula (I), 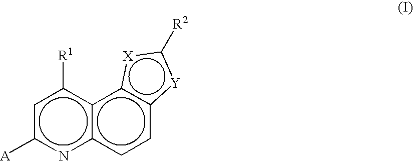
wherein the elements X, Y, A, R¹, R² and R³ have the following meanings:
X denotes a nitrogen atom (N), oxygen atom (O) or sulphur atom (S);
Y denotes a nitrogen atom, if X denotes an oxygen atom or sulphur atom;
Y denotes a nitrogen atom with a bound group R³ or a sulphur atom or an oxygen atom, if X denotes a nitrogen atom;
A denotes an unsubstituted or substituted mono-, di- or tricyclic aromatic group, which contains either no or 1-3 heteroatoms selected from nitrogen, oxygen and sulphur, at least one of the heteroatoms being a nitrogen atom;
R¹ denotes hydroxy, fluorine, chlorine or bromine, amino, (C₁₋₆)alkylamino, di(C₁₋₆)alkylamino, (C₃₋₇)cycloalkylamino, di(C₃₋₇)cycloalkylamino, (C₁₋₆)alkyl-(C₃₋₇)cycloalkylamino, acetidin-1-yl, pyrrolidin-1-yl, pyrrolin-1-yl, imidazolidin-1-yl, imidazolin-1-yl, pyrazolidin-1-yl, pyrazolin-1-yl, piperidin-1-yl, piperazin-1-yl, morpholin-4-yl, thiomorpholin-4-yl, thiomorpholin-S-oxid-4-yl, thiomorpholin-S-dioxid-4-yl, or hexamethyleneimino; and
R² and R³ independently of one another denote hydrogen, (C₁₋₈)alkyl or (C₃₋₇)cycloalkyl,
or a salt thereof.

2. The compound of claim 1, wherein the group A is phenyl, pyridyl, pyrimidyl, pyridazinyl, pyrazinyl, imidazolyl, pyrazolyl, oxazolyl, isoxazolyl, furazanyl, thiazolyl, isothiazolyl or pyrrolyl, unsubstituted or substituted by the groups R⁴, R⁵ and R⁶, where R⁴, R⁵ and R⁶ independently of one another denote hydrogen, (C₁₋₈)alkyl, monofluoro(C₁₋₅)alkyl, difluoro(C₁₋₅)alkyl, trifluoro(C₁₋₅)alkyl, (C₃₋₇)cycloalkyl, hydroxy, (C₁₋₆)alkoxy, fluoromethyloxy, difluoromethyloxy, trifluoromethyloxy, (C₃₋₆)cycloalkyloxy, fluorine, chlorine, bromine, carboxy, (C₁₋₆)alkoxycarbonyl, amino, (C₁₋₆)alkylamino, di(C₁₋₆)alkylamino, acetidin-1-yl, pyrrolidin-1-yl, piperidin-1-yl, (C₁₋₄)acylamino, (C₁₋₆)alkyl-(C₁₋₄)acylamino, aminocarbonyl, (C₁₋₆)alkylaminocarbonyl, di(C₁₋₆)alkylaminocarbonyl, acetidin-1-yl-carbonyl, pyrrolidin-1-yl-carbonyl or piperidin-1-yl-carbonyl.

3. The compound of claim 2, wherein the group A denotes pyridyl or fluorophenyl.

4. The compound of claim 1, wherein the group R¹ denotes amino, methylamino or dimethylamino.

5. The compound of claim 1, wherein the group R² denotes methyl.

6. The compound of claim 1, wherein the group R³ denotes methyl.

7. The compound of claim 1 selected from among the compounds:
3-methyl-9-methylamino-7-(pyridin-4-yl)-3H-imidazo[4,5-f]quinoline;
7-(3-fluorophenyl)-3-methyl-9-methylamino-3H-imidazo[4,5-f]quinoline;
9-dimethylamino-7-(3-fluorophenyl)-3-methyl-3H-imidazo[4,5-f]quinoline;
9-dimethylamino-7-(3-fluorophenyl)-2-methyl-thiazolo[4,5-f]quinoline;
9-dimethylamino-7-(3-fluorophenyl)-thiazolo[5,4-f]quinoline;
7-(3-fluorophenyl)-2-methyl-9-methylamino thiazolo[4,5-f]quinoline;
9-dimethylamino-3-methyl-7-(pyridin-3-yl)-3H-imidazo[4,5-f]quinoline;
3-methyl-9-methylamino-7-(pyridin-3-yl )-3H-imidazo[4, 5-f]quinoline;
2-methyl-9-methylamino-7-(pyridin-3-yl)-thiazolo[4,5-f]quinoline; and
9-dimethylamino-2-methyl-7-(pyridin-3-yl)-thiazolo[4,5-f]quinoline.

8. A process for preparing a compound of claim 1, wherein a 3-oxo-propionic acid ester, the carbonyl group of which is bound to the desired group A, is reacted according to the following reaction plan to give a compound according to the invention, wherein 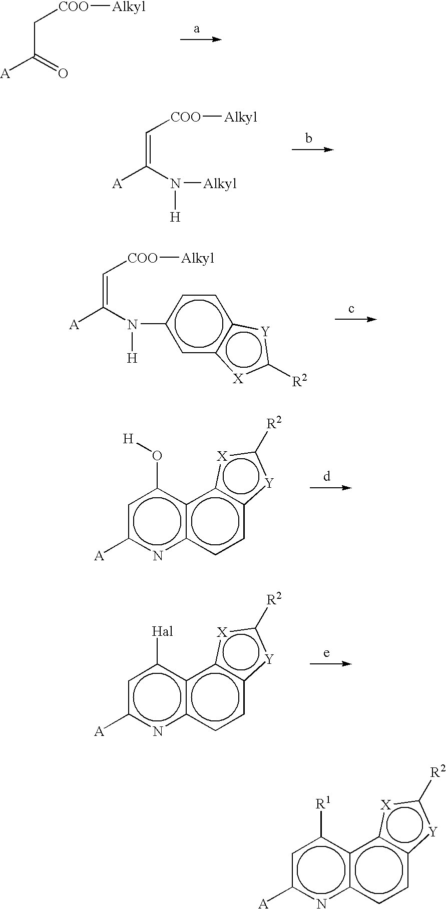
process step a is carried out in the presence of a primary amine;
process step b is carried out in the presence of the desired amino derivative of benzimidazole, benzoxazole or benzthiazole;
process step c is carried out in the presence of a suitable solvent;
process step d is carried out in the presence of a halogenating agent; and
process step e is carried out in the presence of the desired amine.

9. A pharmaceutical composition comprising as an active ingredient a compound of claim 1 and a pharmaceutically acceptable carrier.

10. A method for alleviating or treating pain in a warm blooded animal, comprising administering a therapeutically effective amount of a compound of claim 1.
